(19) United States
(12) Patent Application Publication
(10) Pub. No.: US 2025/0381382 A1
(43) Pub. Date: Dec. 18, 2025

(54) PROTECTION DEVICE FOR MEDICAL ARTICLE

(71) Applicant: Solventum Intellectual Properties Company, Maplewood, MN (US)

(72) Inventors: Ganesh P.D. Kannan, Bangalore (IN); Alan R. Dombrowski, Woodbury, MN (US)

(21) Appl. No.: 19/107,678

(22) PCT Filed: Aug. 17, 2023
(86) PCT No.: PCT/IB2023/058253

§ 371 Date: Feb. 28, 2025

Related U.S. Application Data
(60) Provisional application No. 63/401,961, filed on Aug. 29, 2022.

(51) Int. Cl. A61M 39/20; A61M 5/32; A61M 39/10

(52) U.S. Cl. CPC A61M 39/20; A61M 5/3243; A61M 39/1011; A61M 2205/581; A61M 2205/582

(57) ABSTRACT

A protection device for a medical article is disclosed. The protection device includes an inner housing including a socket disposed at a second inner end thereof, an outer housing, and a tamper indicating assembly disposed proximal to a second outer end of the outer housing. The tamper indicating assembly includes body including a breaking member, and an indicator including an engagement member and a frangible member coupled to the engagement member. Upon at least partially receiving the inner housing within the outer housing, the socket at least partially receives and retains the engagement member therein, such that the engagement member is secured to the inner housing. Upon removal of the inner housing from the outer housing, the breaking member breaks the frangible member. A breakage of the frangible member is indicative of tampering with the protection device.

### TECHNICAL FIELD

The present disclosure generally relates to a protection device for a medical article and a kit including the protection device and the medical article.

### BACKGROUND

Many medical treatments require use of catheters for various purposes, such as feeding, air supply, liquid removal, and/or administering medications and fluids to a patient.

Needleless connectors may connect to an end of the catheters and enable catheter access for infusion and aspiration. The needleless connectors may allow easy access to the catheters, for example, via a port interface thereof. However, this may allow accidental and/or unauthorized access to the catheters via the needleless connectors.

Accidental access to the catheters may lead to infections that can be fatal and/or can severely compromise a recovery time of the patient. Unauthorized access to the catheters may allow drug abusers to introduce narcotic drugs, such as heroin directly into their blood stream.

Therefore, there is a need for a protection device that prevents or reduces accidental and/or intentional unauthorized access to the catheters via the needleless connectors. Furthermore, there is a need for a protection device that ensures an integrity of the needleless connectors and/or the catheters, e.g., by indicating whether the needleless connectors have been tampered with.

### SUMMARY

In a first aspect, the present disclosure provides a protection device for a medical article. The protection device includes an inner housing. The inner housing includes a first inner end and a second inner end opposite to the first inner end. The inner housing further includes a first inner surface defining a first compartment configured to at least partially receive the medical article therein. The inner housing further includes a first outer surface opposite to the first inner surface. The inner housing further includes a socket disposed at the second inner end and on the first outer surface. The protection device further includes an outer housing. The outer housing includes a first outer end and a second outer end opposite to the first outer end. The first outer end is open. The outer housing further includes a second inner surface defining a second compartment configured to at least partially receive the inner housing therein. The outer housing further includes a second outer surface opposite to the second inner surface. The protection device further includes a tamper indicating assembly disposed proximal to the second outer end. The tamper indicating assembly includes a body. The body includes a first body portion coupled to the outer housing at the second outer end. The body further includes a second body portion extending from the first body portion into the second compartment. The body further includes a breaking member extending from the second body portion and spaced apart from the first body portion. The tamper indicating assembly further includes an indicator. The indicator includes at least one engagement member configured to be at least partially received and retained within the socket. The indicator further includes a frangible member coupled to the at least one engagement member and at least partially disposed between the first body portion and the breaking member. The outer housing is configured to at least partially receive the inner housing therein through the first outer end. The inner housing is insertable within the outer housing from the second inner end. Upon at least partially receiving the inner housing within the outer housing, the socket at least partially receives and retains the at least one engagement member therein, such that the at least one engagement member is secured to the inner housing. Upon removal of the inner housing from the outer housing through the first outer end, the breaking member breaks the frangible member of the indicator. A breakage of the frangible member is indicative of tampering with the protection device.

In a second aspect, the present disclosure provides a kit. The kit includes a medical article and the protection device of the first aspect. The protection device at least partially receives and secures the medical article therein. The tamper indicating assembly indicates tampering with at least one of the protection device and the medical article.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIGS. 1A and 1B are schematic top perspective views of medical articles according to different embodiments of the present disclosure;

FIGS. 2A-2C are schematic exploded perspective views of a protection device illustrating the protection device receiving a medical article according to an embodiment of the present disclosure;

FIGS. 3A and 3B are schematic perspective views of the protection device illustrating an insertion of an inner housing within an outer housing of the protection device according to an embodiment of the present disclosure;

FIG. 4 is a schematic partial cross-sectional perspective view of the protection device according to an embodiment of the present disclosure;

FIG. 5 is a schematic cross-sectional perspective view of a portion of the protection device according to an embodiment of the present disclosure;

FIGS. 6A-6C are schematic exploded perspective views of a protection device illustrating the protection device receiving a medical article according to another embodiment of the present disclosure;

FIG. 7 is a schematic perspective view of the protection device of FIGS. 6A-6C according to an embodiment of the present disclosure;

FIGS. 8A and 8B are schematic partial cross-sectional perspective views of a portion of the protection device illustrating a locking engagement according to an embodiment of the present disclosure;

FIGS. 9A-9C are schematic partial cross-sectional perspective views of the protection device illustrating a removal of the inner housing from the outer housing of the protection device according to an embodiment of the present disclosure; and

FIG. 10 is a schematic block diagram of a kit according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials includes less than about 10 weight % of each of the first and second materials.

As used herein, “at least one of A and B” should be understood to mean “only A, only B, or both A and B”.

Unless specified or limited otherwise, the terms “attached,” “connected,” “coupled,” and variations thereof, are used broadly and encompass both direct and indirect attachments, connections, and couplings.

As used herein, the term “configured to” and like is at least as restrictive as the term “adapted to” and requires actual design intention to perform the specified function rather than mere physical capability of performing such a function.

As used herein, the term “spaced apart” refers to elements that are disposed at a distance from one another.

As used herein, the term “medical article” refers to any article that is used for medical purposes on a patient. Medical articles may include catheters, fluid supply and drainage lines, connectors, adaptors, electrical wires and cables, and the like, all of which may be at least partially received within and secured by a protection device of the present disclosure. A medical article may also include needleless connectors that can be connected with medical devices (e.g., catheters), medical supplies, or other medical equipment without using a needle/septum configuration.

As used herein, the term “locking feature” refers to a structure that can engage with a complementary locking feature to lock and prevent relative movement between two components along at least one direction. For example, a first component including a locking feature may be secured to a second component including a complementary locking feature upon locking engagement of the locking feature and the complementary locking feature, such that a relative movement between the first component and the second component along at least one direction is prevented. Such locking features may be designed to permit relative movement between the two components along a first direction while restricting movement along a second direction when engaged. In some cases, the second direction may be opposite to the first direction.

The present disclosure relates to a protection device for a medical article. The protection device includes an inner housing. The inner housing includes a first inner end and a second inner end opposite to the first inner end. The inner housing further includes a first inner surface defining a first compartment configured to at least partially receive the medical article therein. The inner housing further includes a first outer surface opposite to the first inner surface. The inner housing further includes a socket disposed at the second inner end and on the first outer surface. The protection device further includes an outer housing. The outer housing includes a first outer end and a second outer end opposite to the first outer end. The first outer end is open. The outer housing further includes a second inner surface defining a second compartment configured to at least partially receive the inner housing therein. The outer housing further includes a second outer surface opposite to the second inner surface. The protection device further includes a tamper indicating assembly disposed proximal to the second outer end. The tamper indicating assembly includes a body. The body includes a first body portion coupled to the outer housing at the second outer end. The body further includes a second body portion extending from the first body portion into the second compartment. The body further includes a breaking member extending from the second body portion and spaced apart from the first body portion. The tamper indicating assembly further includes an indicator. The indicator includes at least one engagement member configured to be at least partially received and retained within the socket. The indicator further includes a frangible member coupled to the at least one engagement member and at least partially disposed between the first body portion and the breaking member. The outer housing is configured to at least partially receive the inner housing therein through the first outer end. The inner housing is insertable within the outer housing from the second inner end. Upon at least partially receiving the inner housing within the outer housing, the socket at least partially receives and retains the at least one engagement member therein, such that the at least one engagement member is secured to the inner housing. Upon removal of the inner housing from the outer housing through the first outer end, the breaking member breaks the frangible member of the indicator. A breakage of the frangible member is indicative of tampering with the protection device.

The protection device of the present disclosure may at least partially receive and securely retain the medical article therein. Once the socket at least partially receives and retains the at least one engagement member therein, removal of the inner housing from the outer housing may result in the breakage of the frangible member, thereby indicating tampering with the protection device and/or the medical article. Therefore, the protection device may be tamper-evident. The protection device may visually indicate an attempt of tampering with the medical article via the frangible member. The attempt of tampering may be determined by inspecting the frangible member of the indicator. The frangible may be conveniently inspected through an optically transparent window of the outer housing.

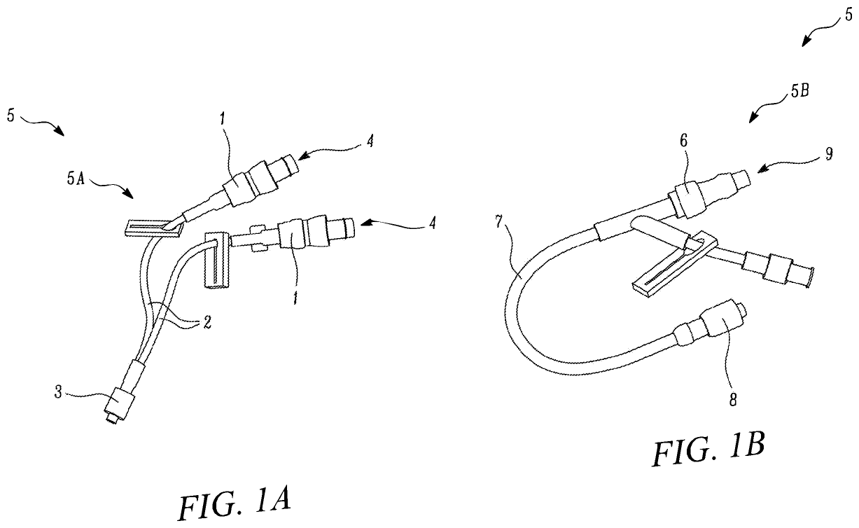

Referring now to the Figures, FIGS. 1A and 1B respectively illustrate schematic top views of medical articles 5 according to different embodiments of the present disclosure.

The medical articles 5 illustrated in FIGS. 1A and 1B may include needleless connectors. The needleless connectors may be connected to an end of catheters and enable catheter access for infusion and aspiration. The needleless connectors may be, for example, simple needleless connectors and complex needleless connectors. The simple needleless connectors may be free of internal moving parts, such as those including an external split septum. The complex needleless connectors may include internal moving components, such as mechanical valves to control a flow of a fluid.

The medical article 5 illustrated in FIG. 1A includes an inline needleless connector assembly 5A. The inline needleless connector assembly 5A may include one or more inline needleless connectors 1, an end port 3, and a tubing 2 that fluidly couples the one or more inline needleless connectors 1 to the end port 3. The one or more inline needleless connectors 1 may include respective port interfaces 4. The end port 3 may be configured to be fluidly connected to a catheter device (not shown). The one or more inline needleless connectors 1 may allow access to the catheter device (e.g., for infusion and/or aspiration) via the respective port interfaces 4. The one or more inline needleless connectors 1 may be simple needleless 30 connectors or complex needleless connectors, as discussed above.

The medical article 5 illustrated in FIG. 1B includes a Y-site needleless connector assembly 5B. The Y-site needleless connector assembly 5B may include a Y-site needleless connector 6, an end port 8, and a tubing 7 that fluidly couples the Y-site needleless connector 6 to the end port 8. The Y-site needleless connector 6 may include a port interface 9. The end port 8 may be configured to be fluidly connected to a catheter device (not shown). The Y-site needleless connector 6 may allow access to the catheter device (e.g., for infusion and/or aspiration) via the port interface 9. The Y-site needleless connector 6 may be a simple needleless connector or a complex needleless connector, as discussed above.

It may be important to prevent accidental access and/or intentional unauthorized access to a catheter device via needleless connectors. For example, it may be important to prevent accidental access and/or intentional unauthorized to the catheter device via the respective port interfaces 4 of the one or more inline needleless connectors 1 and the port interface 9 of the Y-site needleless connector 6.

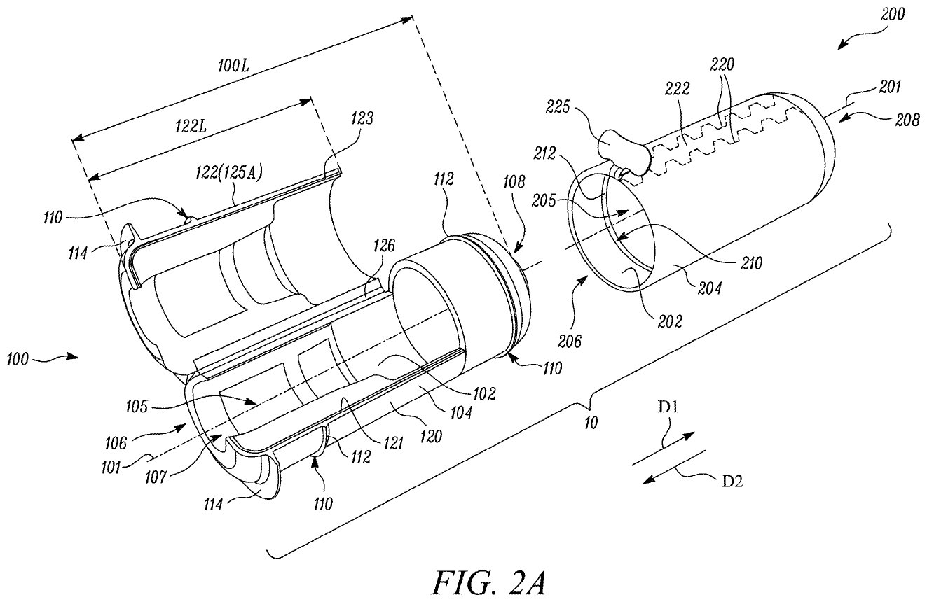

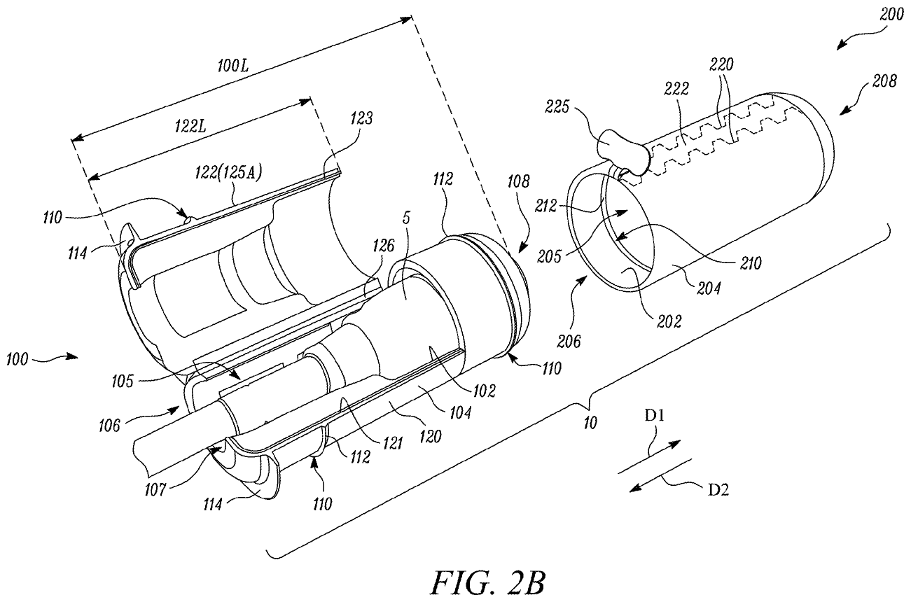

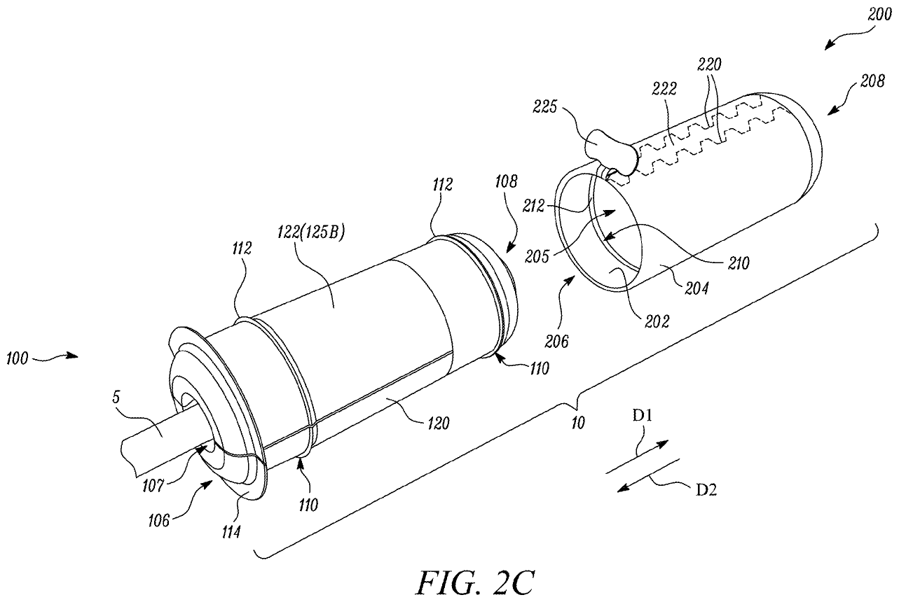

FIGS. 2A-2C illustrate schematic exploded perspective views of a protection device 10 for a medical article (e.g., the medical article 5 of FIG. 1A) according to an embodiment of the present disclosure. Specifically, FIG. 2A illustrates a schematic exploded perspective view of the protection device 10 in a first configuration, FIG. 2B illustrates a schematic exploded perspective view of the protection device 10 in the first configuration with the medical article 5, and FIG. 2C illustrates a schematic exploded perspective view of the protection device 10 in a second configuration with the medical article 5.

Referring to FIGS. 2A-2C, the protection device 10 includes an inner housing 100. The inner housing 100 may extend along an inner longitudinal axis 101. The inner longitudinal axis 101 is shown in FIG. 2A.

The inner housing 100 includes a first inner surface 102 and a first outer surface 104 opposite to the first inner surface 102. The first inner surface 102 defines a first compartment 105 configured to at least partially receive the medical article 5 (shown in FIG. 2B) therein.

In some embodiments, the first inner surface 102 may at least partially conform to a shape of the medical article 5. The medical article 5 may therefore be stably received within the first compartment 105. In other words, the medical article 5 may not jiggle or wobble when received within the first compartment 105.

The inner housing 100 further includes a first inner end 106 and a second inner end 108 opposite to the first inner end 106. The inner housing 100 may extend between the first inner end 106 and the second inner end 108 along the inner longitudinal axis 101.

The second inner end 108 of the inner housing 100 may be closed. The first inner end 106 of the inner housing 100 may be open. Specifically, the inner housing 100 may further include an opening 107 disposed at the first inner end 106. The opening 107 may be configured to at least partially receive the medical article 5 therethrough. For example, as shown in FIG. 2B, a tubing of the medical article 5 may extend through the opening 107 upon insertion of the medical article 5 within the first compartment 105.

The protection device 10 further includes an outer housing 200. The outer housing 200 may extend along an outer longitudinal axis 201. The outer longitudinal axis 201 is shown in FIG. 2A. The outer longitudinal axis 201 may be parallel to the inner longitudinal axis 101 of the inner housing 100.

The outer housing 200 includes a second inner surface 202 and a second outer surface 204 opposite to the second inner surface 202. The second inner surface 202 defines a second compartment 205 configured to at least partially receive the inner housing 100 therein. The second inner surface 202 may at least partially conform to a shape of the inner housing 100. Specifically, the second inner surface 202 of the outer housing 200 may at least partially conform to the first outer surface 104 of the inner housing 100.

The inner housing 100 and the outer housing 200 may any suitable complementary shape, such as, cylindrical, rectangular, polygonal, etc. The second inner surface 202 of the outer housing 200 may have greater dimensions than the first outer surface 104 of the inner housing 100, with suitable tolerances for manufacturing.

The inner housing 100 and the outer housing 200 may be made from any suitable material or materials, such as polymeric materials (e.g., acrylonitrile butadiene styrene (ABS), polycarbonate, etc.). The inner housing 100 and the outer housing 200 may be manufactured utilizing any suitable technique or techniques (e.g., molding, 3D printing, etc.). In some embodiments, each of the inner housing 100 and the outer housing 200 may be a single-piece molded housing made from, for example, injection molding of polymeric materials.

The outer housing 200 further includes a first outer end 206 and a second outer end 208 opposite to the first outer end 206. The outer housing 200 may extend between the first outer end 206 and the second outer end 208 along the outer longitudinal axis 201. The second outer end 208 of the outer housing 200 may be closed. The first outer end 206 is open.

The outer housing 200 is configured to at least partially and slidably receive the inner housing 100 therein through the first outer end 206 along a first direction DI from the first outer end 206 to the second outer end 208. The first direction D1 may be substantially parallel to each of the inner longitudinal axis 101 of the inner housing 100 and the outer longitudinal axis 201 of the outer housing 200. The inner housing 100 is insertable within the outer housing 200 from the second inner end 108.

It may be noted that the first direction DI is defined with the consideration that the inner housing 100 is moved relative to the outer housing 200. Alternatively, with the consideration that the outer housing 200 is moved relative to the inner housing 100, the first direction may extend from the second outer end 208 toward the first outer end 206, i.e., opposite to what is discussed above.

For the purposes of clarity and consistency, when the first direction DI is referenced in the present disclosure, it is to be understood that the inner housing 100 is contemplated to be moved relative to the outer housing 200 in the first direction DI from the first outer end 206 to the second outer end 208. That is, the inner housing 100 is contemplated to be moved along the first direction DI relative to the outer housing 200, such that the inner housing 100 is inserted within the outer housing 200 from the second inner end 108.

The inner housing 100 may further include a primary portion 120 and a lid portion 122 movably coupled to the primary portion 120. The lid portion 122 may be movable relative to the primary portion 120 between an open position 125A (shown in FIGS. 2A and 2B) and a closed position 125B (shown in FIG. 2C).

As shown in FIG. 2B, the inner housing 100 may be configured to at least partially receive the medical article 5 therein in the open position 125A of the lid portion 122. In other words, the first compartment 105 may be accessible in the open position 125A of the lid portion 122, such that the medical article 5 can be inserted therein. Furthermore, as shown in FIG. 2C, the medical article 5 may be secured within the inner housing 100 in the closed position 125B of the lid portion 122.

The inner housing 100 may further include an inner housing length 100L. The inner housing length 100L may be defined between the first inner end 106 and the second inner end 108 of the inner housing 100 along the inner longitudinal axis 101. Further, the lid portion 122 may include a lid length 122L. The lid length 122L may be greater than 50% of the inner housing length 100L. A greater lid length 122L (e.g., greater than 50% of the inner housing length 100L) may facilitate insertion of the medical article 5 within the first compartment 105. In some embodiments, the lid length 122L may be greater than 60%, greater than 70%, greater than 80%, or greater than 90% of the inner housing length 100L.

The inner housing 100 may further include a hinge 126 pivotally coupling the lid portion 122 to the primary portion 120. In other words, the primary portion 120 and the lid portion 122 may be pivotally coupled to each other by the hinge 126. The lid portion 122 may therefore be pivotable between the open position 125A and the closed position 125B about the hinge 126. In some embodiments, the hinge 126 may be a living hinge. The hinge 126 may be a living hinge, for example, when the inner housing 100 is injection molded as a single piece.

The primary portion 120 may include a first lid locking feature 121. The lid portion 122 may include a second lid locking feature 123 complementary to the first lid locking feature 121. The first lid locking feature 121 may lockingly engage with the second lid locking feature 123 in the closed position 125B (shown in FIG. 2C) of the lid portion 122, such that the primary portion 120 and the lid portion 122 are secured to each other. In some embodiments, a locking engagement of the second lid locking feature 123 with the first lid locking feature 121 may retain the lid portion 122 in the closed position 125B.

As shown in FIGS. 2A and 2B, the second lid locking feature 123 may include a projection and the first lid locking feature 121 may include a recess configured to lockingly receive the projection therein. Alternatively, although not illustrated, the first lid locking feature 121 may include a projection and the second lid locking feature 123 may include a recess configured to lockingly receive the projection therein. The first lid locking feature 121 and the second lid locking feature 123 may together form a snap-fit connection. The snap-fit connection may facilitate securement of the lid portion 122 to the primary portion 120 in the closed position 125B of the lid portion 122. Furthermore, the snap-fit connection may provide a tactile and/or an audible indication when the primary portion 120 and the lid portion 122 are secured to each other.

The inner housing 100 further includes at least one inner locking feature 110 disposed on the first outer surface 104. The outer housing 200 further includes at least one outer locking feature 210 disposed on the second inner surface 202. The at least one outer locking feature 210 is complementary to the at least one inner locking feature 110. Specifically, the at least one outer locking feature 210 is configured to lockingly engage with the at least one inner locking feature 110.

As shown in FIGS. 2A and 2B, the at least one inner locking feature 110 may be partially disposed on the primary portion 120 and partially disposed on the lid portion 122. The primary portion 120 and the lid portion 122 may together define the at least one inner locking feature 110 in the closed position 125B (shown in FIG. 2C) of the lid portion 122.

As shown in FIGS. 2A-2C, the at least one inner locking feature 110 may include a protrusion 112 extending from the first outer surface 104 opposite to the first inner surface 102. Further, the at least one outer locking feature 210 may include a recess 212 extending partially from the second inner surface 202 toward the second outer surface 204 and configured to at least partially receive the protrusion 112 therein. Alternatively, although not illustrated, the at least one outer locking feature 210 may include a protrusion (e.g., similar to the protrusion 112) extending from the second inner surface 202 opposite to the second outer surface 204, and the at least one inner locking feature 110 may include a recess (e.g., similar to the recess 212) extending partially from the first outer surface 104 toward the first inner surface 102 and configured to at least partially receive the protrusion therein.

In the illustrated embodiment of FIGS. 2A-2C, each of the inner housing 100 and the outer housing 200 has a substantially cylindrical shape. The protrusion 112 may circumferentially extend around the first outer surface 104 of the inner housing 100. The recess 212 may circumferentially extend around the second inner surface 202 of the outer housing 200.

In some embodiments, the at least one inner locking feature 110 (or the protrusion 112) may have a circumferential extent of 360 degrees in the closed position 125B (shown in FIG. 2C) of the lid portion 122. Moreover, the at least one outer locking feature 210 (or the recess 212) may have a circumferential extent of 360 degrees. This may allow the at least one inner locking feature 110 to lockingly engage with the at least one outer locking feature 210 in any relative rotational configuration of the inner housing 100 and the outer housing 200.

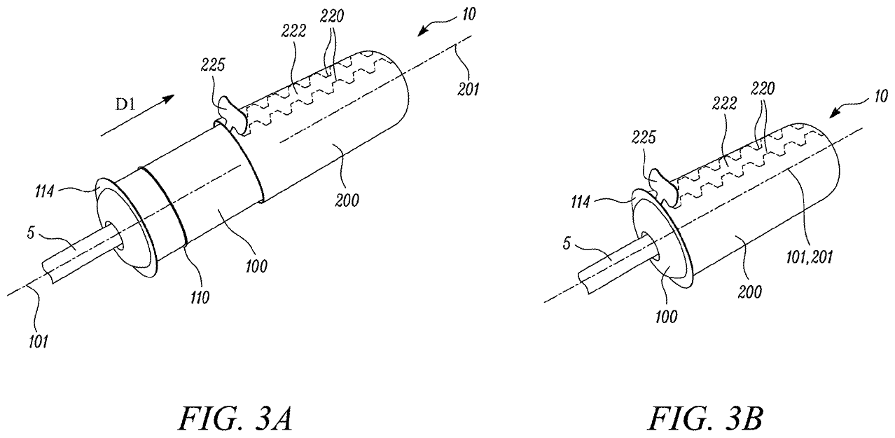

FIGS. 3A and 3B schematically illustrate an insertion of the inner housing 100 within the outer housing 200 along the first direction D1. Specifically, FIG. 3A illustrates a first step of the insertion of the inner housing 100 within the outer housing 200 and FIG. 3B illustrates a second step of the insertion of the inner housing 100 within the outer housing 200. The second step illustrated in FIG. 3B may be a final step of the insertion.

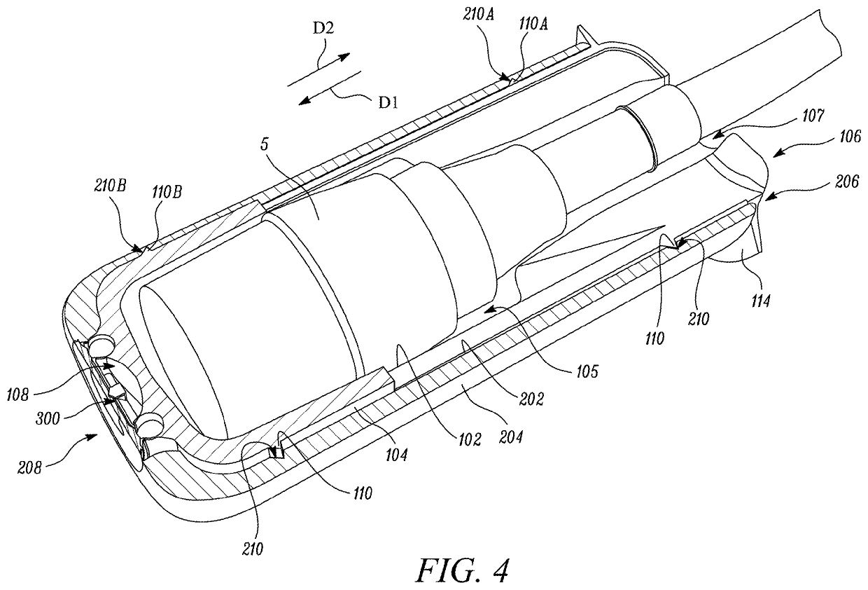

FIG. 4 illustrates a schematic partial cross-sectional perspective view of the protection device 10 in the second step shown in FIG. 3B. The medical article 5 is also shown in FIG. 4.

Referring to FIGS. 3A-4, upon insertion of the inner housing 100 within the outer housing 200 along the first direction D1, the at least one inner locking feature 110 lockingly engages with the at least one outer locking feature 210. Further, upon insertion of the inner housing 100 within the outer housing 200, the first outer surface 104 may at least partially engage with the second inner surface 202.

As shown in FIG. 3B, upon insertion of the inner housing 100 within the outer housing 200 along the first direction D1, the outer longitudinal axis 201 may be coaxial to the inner longitudinal axis 101. In other words, the inner housing 100 may be coaxially inserted within the outer housing 200, such that the outer longitudinal axis 201 becomes coaxial to the inner longitudinal axis 101.

Upon locking engagement of the at least one inner locking feature 110 with the at least one outer locking feature 210, the inner housing 100 is secured to the outer housing 200, such that the inner housing 100 is prevented from moving relative to the outer housing 200 along a second direction D2 opposite to the first direction D1. The second direction D2 is also shown in FIGS. 2A-2C. The second direction D2 may be substantially parallel to each of the inner longitudinal axis 101 and the outer longitudinal axis 201. Furthermore, upon locking engagement of the at least one inner locking feature 110 with the at least one outer locking feature 210, the outer housing 200 may at least partially enclose the lid portion 122 and retain the lid portion 122 in the closed position 125B.

As shown in FIG. 4, the at least one inner locking feature 110 may include a first inner locking feature 110A proximal to the first inner end 106 and a second inner locking feature 110B distal to the first inner end 106. The second inner locking feature 110B may be proximal to the second inner end 108. Further, the at least one outer locking feature 210 may include a first outer locking feature 210A proximal to the first outer end 206 and a second outer locking feature 210B distal to the first outer end 206. The second outer locking feature 210B may be proximal to the second outer end 208.

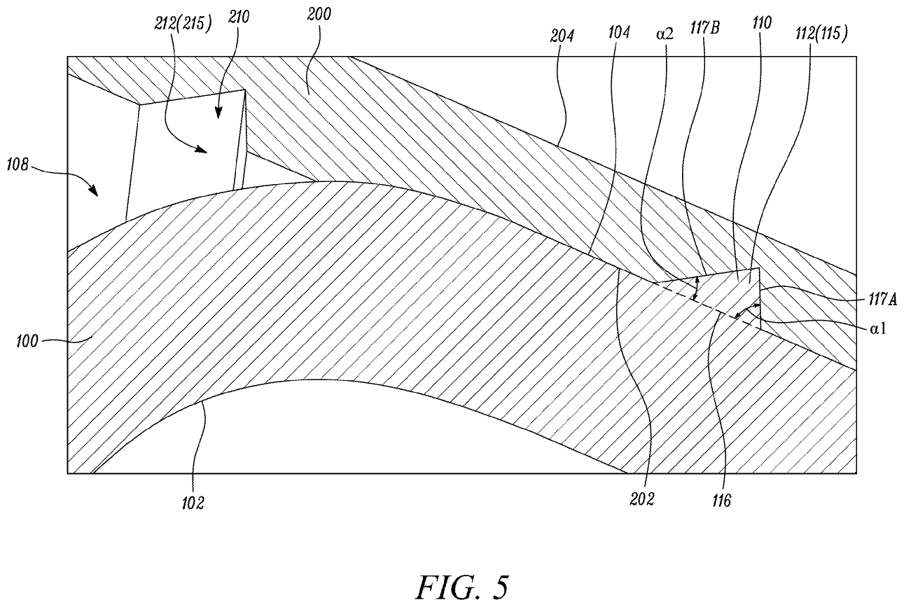

During insertion of the inner housing 100 within the outer housing 200 along the first direction D1, the second inner locking feature 110B may slide over the first outer locking feature 210A. The second inner locking feature 110B may slide over the first outer locking feature 210A as the inner housing 100 is inserted within the outer housing 200 from the first step of the insertion shown in FIG. 3A to the second step of the insertion shown in FIG. 3B. The second inner locking feature 110B may slide over the first outer locking feature 210A due to respective designs of the protrusion 112 and the recess 212. Examples of such designs will be discussed in detail later with reference to FIG. 5.

Upon locking engagement of the first inner locking feature 110A with the first outer locking feature 210A, the second inner locking feature 110B may lockingly engage with the second outer locking feature 210B. This may be achieved at the second step of the insertion shown in FIGS. 3B and 4. Upon locking engagement of the first inner locking feature 110A with the first outer locking feature 210A and the second inner locking feature 110B with the second outer locking feature 210B, the inner housing 100 may be prevented from moving relative to the outer housing 200 along the second direction D2.

The inner housing 100 may further include a flange 114 disposed proximal to the first inner end 106. The flange 114 may extend from the first outer surface 104 opposite to the first inner surface 102. The flange 114 may extend circumferentially around the first outer surface 104 of the inner housing 100.

As shown in FIG. 4, upon locking engagement of the at least one inner locking feature 110 with the at least one outer locking feature 210, the outer housing 200 may engage with the flange 114 to prevent further movement of the outer housing 200 relative to the inner housing 100 along the first direction D1. The flange 114 may have greater dimensions (e.g., a greater maximum diameter) than the outer housing 200. Advantageously, upon locking engagement of the at least one inner locking feature 110 with the at least one outer locking feature 210, the flange 114 may prevent prying of the outer housing 200 from the first outer end 206.

As shown in FIGS. 2A and 2B, the flange 114 may be partially disposed on the primary portion 120 and partially disposed on the lid portion 122. The primary portion 120 and the lid portion 122 may together define the flange 114 in the closed position 125B (shown in FIG. 2C) of the lid portion 122.

The protection device 10 may produce a locking feedback upon locking engagement of the at least one inner locking feature 110 with the at least one outer locking feature 210. The locking feedback may include at least one of an audible feedback and a tactile feedback. The locking feedback may be produced by virtue of locking engagement of at least one inner locking feature 110 with the at least one outer locking feature 210. The locking feedback may aid in ensuring that locking engagement of the at least one inner locking feature 110 with the at least one outer locking feature 210 is achieved.

In some cases, the protection device 10 may produce the locking feedback when the second inner locking feature 110B slides over the first outer locking feature 210A. In such cases, the protection device 10 may produce two instances of the locking feedback, once when the second inner locking feature 110B slides over the first outer locking feature 210A, and once when the when the first inner locking feature 110A lockingly engages with the first outer locking feature 210A.

It may be noted that once the at least one outer locking feature 210 (or the second outer locking feature 210B) of the outer housing 200 lockingly engages with the at least one inner locking feature 110 (or the second inner locking feature 110B) of the inner housing 100, the inner housing 100 may not be removable from the outer housing 200 without damaging the outer housing 200.

Now referring to FIGS. 2A-3B, the outer housing 200 may further include a pair of lines of weakness 220 extending along a length of the outer housing 200. The pair of lines of weakness 220 may define a tear region 222 therebetween. The outer housing 200 may further include a tab 225 coupled to the tear region 222 and extending outwardly relative to the second outer surface 204.

At least the tear region 222 may be configured to separate from the rest of the outer housing 200 upon application of force on the tab 225 at least in the first direction D1. The tab 225 may be grasped and pulled (e.g., in the first direction D1 shown in FIG. 3A) to separate the tear region 222 from the rest of the outer housing 200. Upon separation of the tear region 222 from the rest of the outer housing 200, the inner housing 100 may be removed from the outer housing 200. Therefore, the protection device 10 may be tamper evident and indicate tampering with the protection device 10 and/or the medical article 5. For example, the tear region 222 of the outer housing 200 may be observed to determine an attempt of tampering with the protection device 10 and/or the medical article 5.

The protection device 10 may further include a tamper indicating assembly 300 (shown in FIG. 4) disposed proximal to the second outer end 208 of the outer housing 200. The tamper indicating assembly 300 may additionally or alternatively indicate an attempt of tampering with the protection device 10 and/or the medical article 5. The tamper indicating assembly 300 will be discussed in detail later.

FIG. 5 illustrates a schematic cross-sectional perspective view of a portion of the protection device 10 according to an embodiment of the present disclosure.

As shown in FIG. 5, the protrusion 112 may include a triangular cross-sectional shape 115. The recess 212 may include a complementary triangular cross-sectional shape 215. In other words, the triangular cross-sectional shape 215 of the recess 212 may be complementary to the triangular cross-sectional shape 115 of the protrusion 112.

Referring to FIGS. 3A-5, the first outer surface 104 may correspond to a base 116 of the triangular cross-sectional shape 115. The other two sides 117A, 117B of the triangular cross-sectional shape 115 may define different respective angles α1, α2 with respect to the base 116, such that upon locking engagement of the at least one inner locking feature 110 with the at least one outer locking feature 210, the inner housing 100 is slidable relative to the outer housing 200 along the first direction D1 and the inner housing 100 is prevented from moving relative to the outer housing 200 along the second direction D2.

The angle al may be greater than the angle α2. Specifically, the angle α2 defined by the side 117B of the triangular cross-sectional shape 115 proximal to the second inner end 108 of the inner housing 100 may be between 30% to 80% of the angle al defined by the side 117A of the triangular cross-sectional shape 115 distal to the second inner end 108 of the inner housing 100.

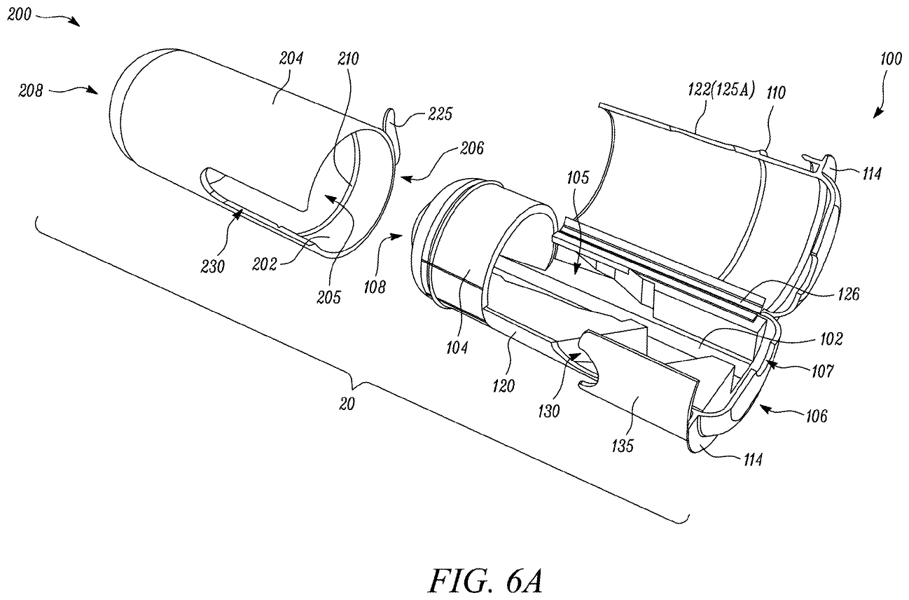

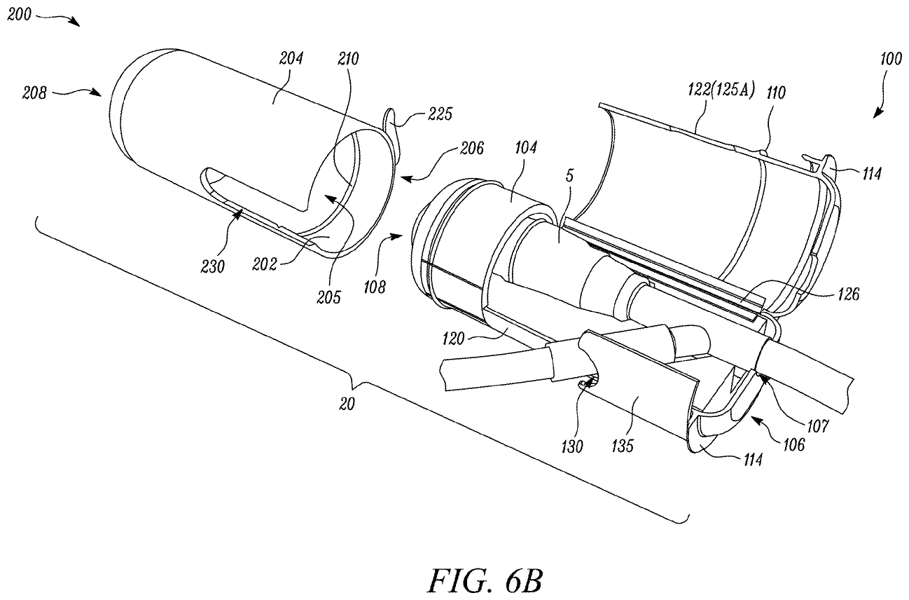

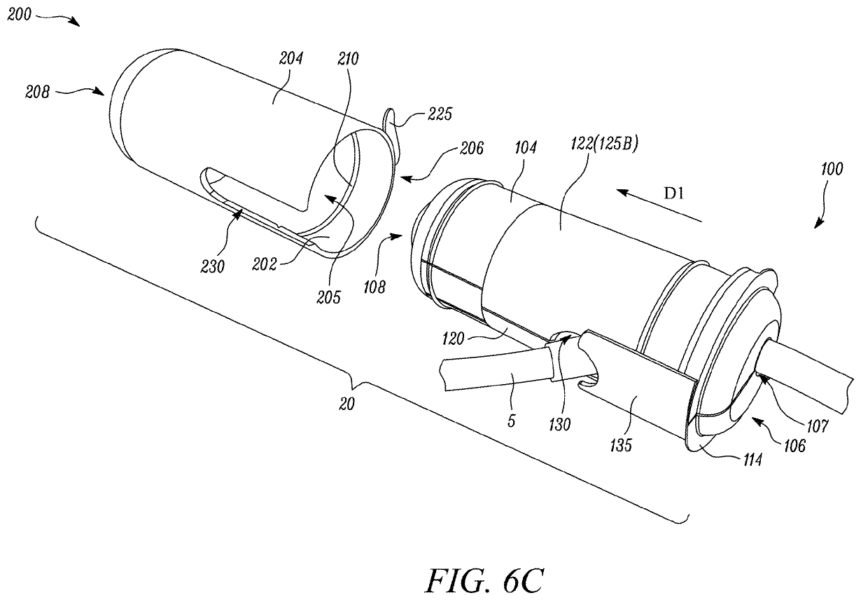

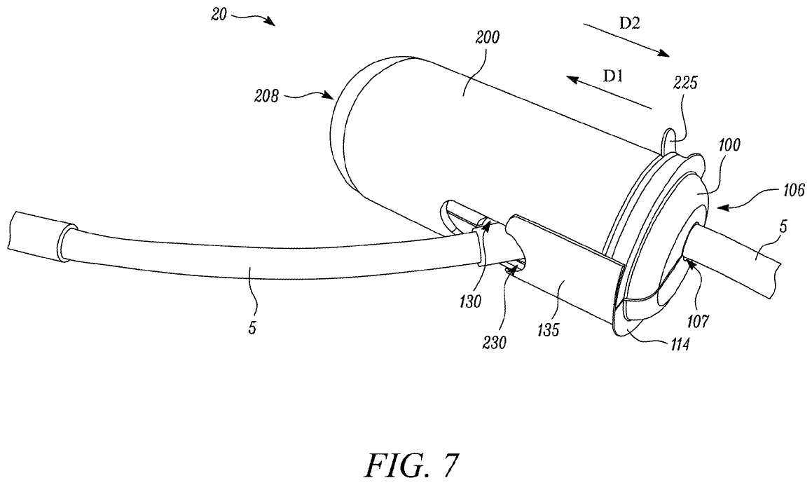

FIGS. 6A-6C illustrate schematic exploded perspective views of a protection device 20 for a medical article (e.g., the medical article 5 of FIG. 1B) according to another embodiment of the present disclosure. Specifically, FIG. 6A illustrates a schematic exploded perspective view of the protection device 20 in a first configuration, FIG. 6B illustrates a schematic exploded perspective view of the protection device 20 in the first configuration with the medical article 5, and FIG. 6C illustrates a schematic exploded perspective view of the protection device 20 in a second configuration with the medical article 5. Further, FIG. 7 illustrates a schematic perspective view of the protection device 20.

The protection device 20 is similar to the protection device 10 of FIGS. 2A-2C, with like elements designated by like numbers. However, the inner housing 100 and the outer housing 200 of the protection device 20 include different designs than those of the protection device 10 to accommodate a different type of the medical article 5.

Referring to FIGS. 6A-6C, the inner housing 100 may further include a first cutout 130. The outer housing 200 may further include a second cutout 230.

The first cutout 130 may extend from the first inner surface 102 to the first outer surface 104. Therefore, the first cutout 130 may be a through-cutout extending through the inner housing 100. The first cutout 130 may be partially disposed on the primary portion 120 of the inner housing 100 and partially disposed on the lid portion 122 of the inner housing 100. As shown in FIG. 6C, the primary portion 120 and the lid portion 122 may together define the first cutout 130 in the closed position 125B of the lid portion 122.

The second cutout 230 may extend from the second inner surface 202 to the second outer surface 204. Therefore, the second cutout 230 may be a through-cutout extending through the outer housing 200. Further, the second cutout 230 may partially extend from the first outer end 206 toward the second outer end 208. Thus, the second cutout 230 may be a through-slot partially extending from the first outer end 206 toward the second outer end 208. This may allow insertion of the inner housing 100 within the outer housing 200 when the medical article 5 is secured within the inner housing 100, as shown in FIG. 6B.

Upon insertion of the inner housing 100 within the outer housing 200, the first cutout 130 may be at least partially aligned with the second cutout 230. Furthermore, as shown in FIG. 7, the first cutout 130 and the second cutout 230 may together at least partially receive the medical article 5 therethrough. The configuration of the inner housing 100 and the outer housing 200 of the protection device 20 may be suitable for use with Y-site needleless connectors, such as the Y-site needleless connector 6 shown in FIG. 1B.

In some embodiments, the inner housing 100 may further include an information tab 135 disposed proximal to the first inner end 106. The first cutout 130 may further be partially defined by the information tab 135. The information tab 135 may be at least partially spaced apart from the first outer surface 104. In some embodiments, a portion of the information tab 135 may extend from the first outer surface 104 opposite to the first inner surface 102. In some embodiments, the information tab 135 may be attached to the flange 114.

The information tab 135 may be utilized to display an information regarding the medical article 5 and/or the protection device 20. For example, the information tab 135 may be utilized to display that the protection device 20 is tamper evident. In some cases, the information tab 135 may be utilized to display a patient information, such as details of the patient, details of a medical treatment, such as a medicament to be used with the medical article, and the like. The information may be displayed on the information tab 135 by any suitable technique. For example, the information may be written on the information tab 135 (e.g., with a permanent ink), printed on the information tab 135, printed on a sticker that is attached to the information tab 135, inscribed on the information tab 135, and the like.

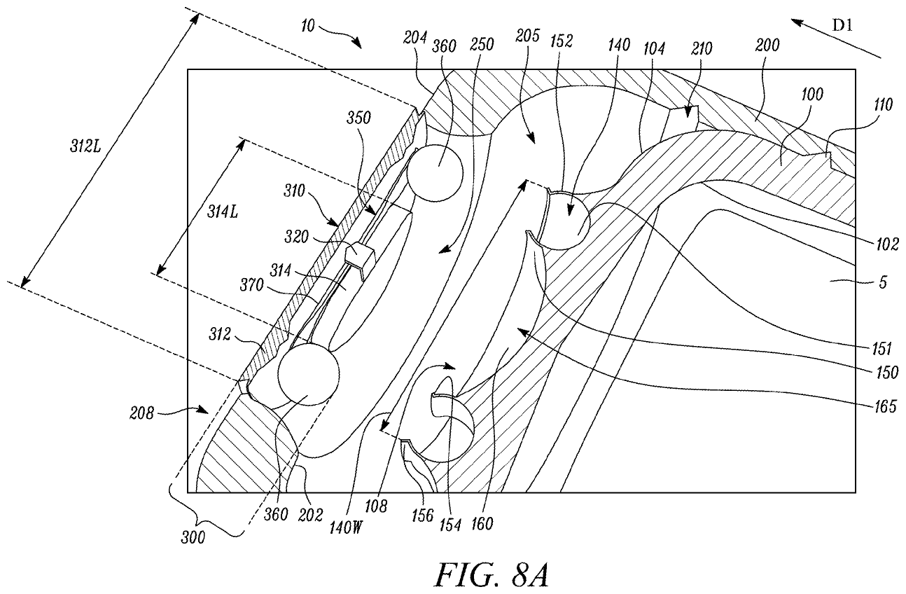

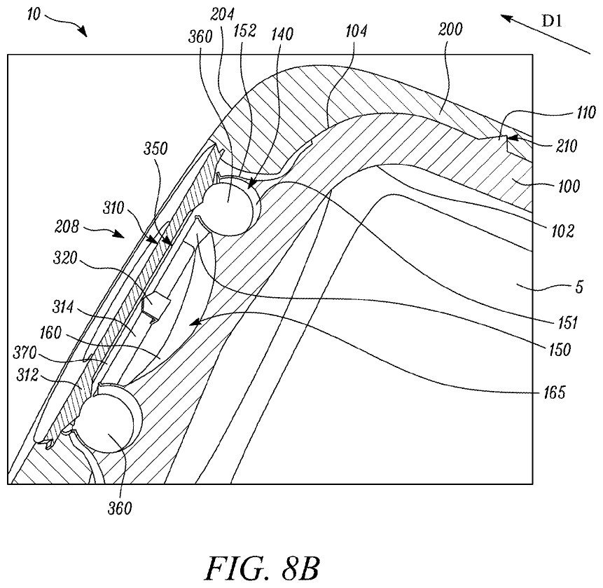

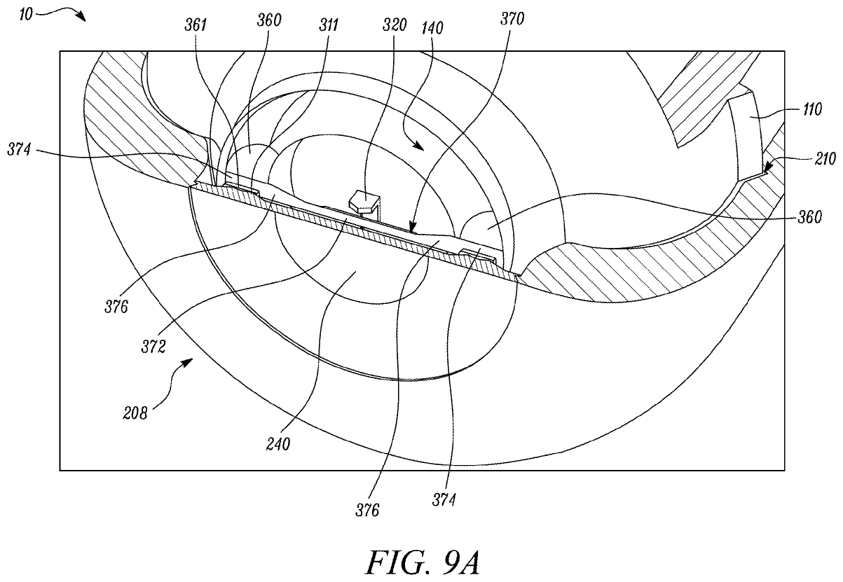

FIGS. 8A and 8B illustrate schematic partial cross-sectional perspective views of a portion of the protection device 10 according to an embodiment of the present disclosure. Specifically, FIG. 8A illustrates a schematic partial cross-sectional perspective view of a portion of the protection device 10 prior to a locking engagement of the at least one inner locking feature 110 and the at least one outer locking feature 210. FIG. 8B illustrates a schematic partial cross-sectional perspective view of a portion of the protection device 10 subsequent to a locking engagement of the at least one inner locking feature 110 and the at least one outer locking feature 210.

Referring to FIGS. 8A and 8B, the protection device 10 may further include the tamper indicating assembly 300 disposed proximal to the second outer end 208 of the outer housing 200.

The tamper indicating assembly 300 may include a body 310. The body 310 may include a first body portion 312 and a second body portion 314 extending from the first body portion 312 into the second compartment 205.

The first body portion 312 may be coupled to the outer housing 200 at the second outer end 208. In some embodiments, the first body portion 312 may be rotatably coupled to the outer housing 200, such that the body 310 of the tamper indicating assembly 300 is rotatable relative to the outer housing 200. The second body portion 314 may be smaller in dimensions than the first body portion 312. In some embodiments, a maximum length 314L of the second body portion 314 may be between 30% to 80% of a maximum length 312L of the first body portion 312.

The body 310 may further include a breaking member 320 extending from the second body portion 314. The breaking member 320 may be spaced apart from the first body portion 312. Therefore, the breaking member 320 and the first body portion 312 may define a gap therebetween.

The tamper indicating assembly 300 may further include an indicator 350. The indicator 350 may include at least one engagement member 360 and a frangible member 370 coupled to the at least one engagement member 360. The frangible member 370 may be at least partially disposed between the first body portion 312 and the breaking member 320. That is, the frangible member 370 may be at least partially disposed in the gap between the first body portion 312 and the breaking member 320.

As shown in FIG. 8A, the inner housing 100 may further include a socket 140 disposed at the second inner end 108 and on the first outer surface 104. The socket 140 may partially extend from the first outer surface 104 toward the first inner surface 102. The socket 140 may include a maximum width 140W. In some embodiments, the maximum width 140W of the socket 140 may be less than the maximum length 312L of the first body portion 312.

As shown in FIG. 8B, the at least one engagement member 360 may be configured to be at least partially received and retained within the socket 140. In other words, the socket 140 may lockingly receive the at least one engagement member 360 therein. The at least one engagement member 360 may snap-fit into the socket 140.

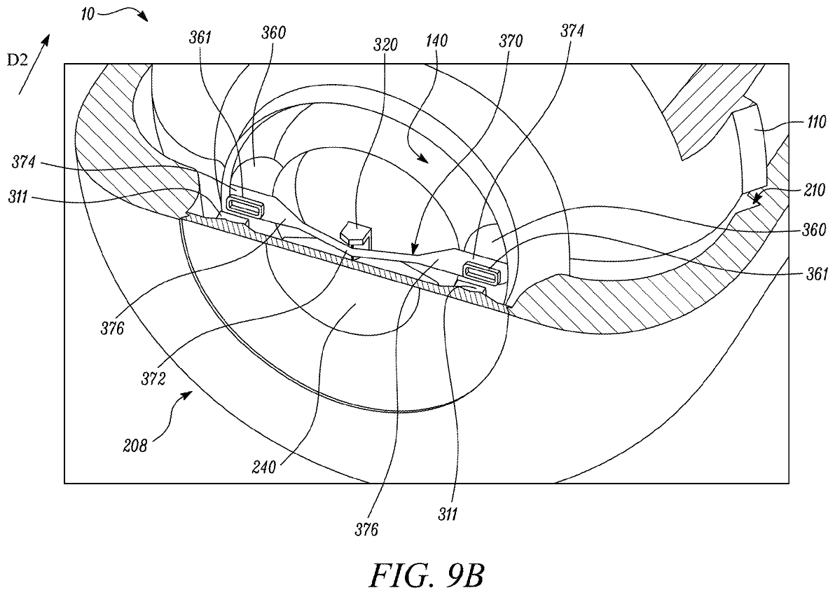

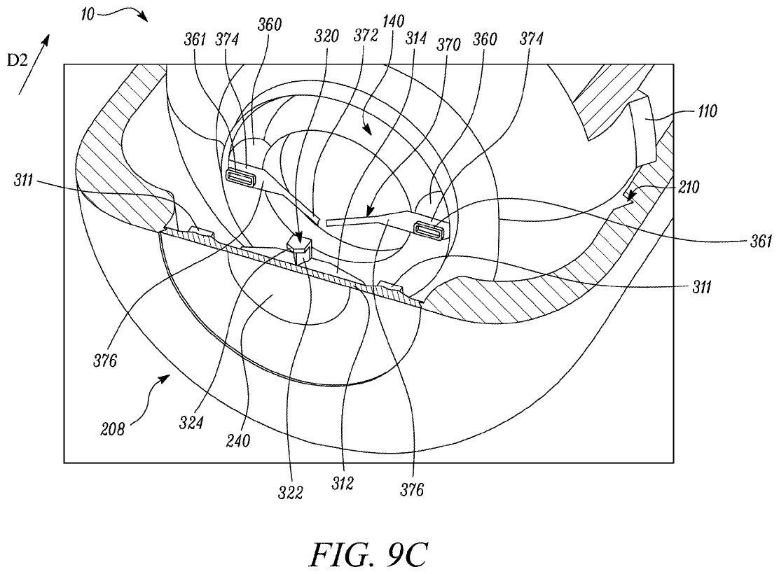

The at least one engagement member 360 may include a spherical shape. The socket 140 may have a circular cross-sectional shape. The circular cross-sectional shape of the socket 140 may be complementary to the spherical shape of the at least one engagement member 360, such that the at least one engagement member 360 can be at least partially received and retained within the socket 140. Furthermore, the socket 140 may have an annular shape (as shown in FIG. 9A). The annular shape may enable the socket 140 to at least partially receive the at least one engagement member 360 in any relative rotational configuration of the inner housing 100 and the outer housing 200.

The inner housing 100 may further include an inner annular wall 150 disposed at the second inner end 108, an outer annular wall 152 disposed at the second inner end 108 and surrounding the inner annular wall 150, and an end wall 151 connecting the inner annular wall 150 to the outer annular wall 152. Each of the inner annular wall 150, the outer annular wall 152, and the end wall 151 may be curved. The socket 140 may be defined between the outer annular wall 152, the inner annular wall 150, and the end wall 151. In other words, the outer annular wall 152, the inner annular wall 150, and the end wall 151 may together define the socket 140.

The inner annular wall 150 may include an inwardly extending lip 154 at an end thereof. The outer annular wall 152 may include an outwardly extending lip 156 at an end thereof. The inwardly extending lip 154 and the outwardly extending lip 156 may facilitate insertion of the at least one engagement member 360 within the socket 140. Specifically, upon insertion of the at least one engagement member 360, the inwardly extending lip 154 may facilitate an inward elastic deformation of the inner annular wall 150, and the outwardly extending lip 156 may facilitate an outward elastic deformation of the outer annular wall 152, such that the at least one engagement member 360 is at least partially received within the socket 140.

The inner housing 100 may further include a concave wall 160 surrounded by the inner annular wall 150. The concave wall 160 and the inner annular wall 150 may together define a recess 165 therebetween. As shown in FIG. 8B, the recess 165 may at least partially receive the second body portion 314 therein upon insertion of the at least one engagement member 360 into the socket 140.

The outer housing 200 may further include an outer recess 250 (shown in FIG. 8A) at the second outer end 208. The outer recess 250 may extend from the second inner surface 202 at least partially toward the second outer surface 204. The indicator 350 may be at least partially received within the outer recess 250.

As shown in FIG. 8B, upon at least partially receiving the inner housing 100 within the outer housing 200, the socket 140 may at least partially receive and retain the at least one engagement member 360 therein, such that the at least one engagement member 360 is secured to the inner housing 100. For example, upon locking engagement of the at least one inner locking feature 110 with the at least one outer locking feature 210, the socket 140 may at least partially receive and retain the at least one engagement member 360 therein, such that the at least one engagement member 360 is secured to the inner housing 100.

FIGS. 9A-9C illustrate schematic partial cross-sectional perspective views of a portion of the protection device 10 according to an embodiment of the present disclosure. FIGS. 9A-9C schematically illustrate removal of the inner housing 100 from the outer housing 200 and an effect thereof on the tamper indicating assembly 300.

Referring to FIGS. 9A-9C, upon removal of the inner housing 100 from the outer housing 200 through the first outer end 206, the breaking member 320 may break the frangible member 370 of the indicator 350. A breakage of the frangible member 370 may be indicative of tampering with the protection device 10. The at least one engagement member 360 may be secured and retained in the socket 140 after the breakage of the frangible member 370.

In other words, once the at least one engagement member 360 is secured to the inner housing 100 via the socket 140, removal of the inner housing 100 from the outer housing 200 may cause the breaking member 320 to break the frangible member 370, thereby indicating tampering with the protection device 10. The outer housing 200 may include an optically transparent window 240 at the second outer end 208, such that at least the frangible member 370 is visible through the optically transparent window 240. Therefore, the frangible member 370 may be visible and observed through the optically transparent window 240. An unbroken state of the frangible member 370 may indicate that the protection device 10 and/or the medical article 5 is not tampered with. A broken state of the frangible member 370 may indicate an attempt of tampering with the protection device 10 and/or the medical article 5.

The at least one engagement member 360 may include a first engagement feature 361 (best shown in FIGS. 9B and 9C). The body 310 may further include a second engagement feature 311 complementary to the first engagement feature 361 (best shown in FIGS. 9B and 9C).

The second engagement feature 311 may be configured to releasably engage with the first engagement feature 361. Prior to the at least one engagement member 360 being received by the socket 140 (e.g., as shown in FIG. 8A), the first engagement feature 361 may be releasably engaged with the second engagement feature 311, such that the at least one engagement member 360 is releasably secured to the body 310 of the tamper indicating assembly 300.

As discussed above, the socket 140 may at least partially receive and retain the at least one engagement member 360 therein, such that the at least one engagement member 360 is secured to the inner housing 100. As shown in FIGS. 9B and 9C, upon removal of the inner housing 100 from the outer housing 200 through the first outer end 206 (shown in FIG. 4), the first engagement feature 361 may detach from the second engagement feature 311. In other words, upon movement of the inner housing 100 in the second direction D2, the first engagement feature 361 may detach from the second engagement feature 311.

As shown in FIGS. 9B and 9C, the first engagement feature 361 may include an engagement socket. Further, the second engagement feature 311 may include an engagement projection configured to be at least partially and releasably received within the engagement socket.

The at least one engagement member 360 may include a pair of engagement members 360 spaced apart from each other and coupled to the frangible member 370 at opposite ends thereof.

The frangible member 370 may include a narrow portion 372 configured to be engaged and broken by the breaking member 320. The frangible member 370 may further include a pair of wide portions 374. Each of the pair of wide portions 374 may be connected to a respective engagement member 360 from the pair of engagement members 360. Further, the first engagement feature 361 may be disposed on each of the pair of wide portions 374. The frangible member 370 may further include a pair of tapered portions 376. Each of the pair of tapered portions 376 may connect the narrow portion 372 to a respective wide portion 374 from the pair of wide portions 374.

Further, as shown in FIG. 9C, the breaking member 320 may include a breaking portion 322 extending from the second body portion 314. The breaking portion 322 may include a triangular cross-sectional shape. The triangular cross-sectional shape may facilitate a breakage of the frangible member 370. Specifically, the triangular cross-sectional shape may facilitate a breaking of the narrow portion 372 of the frangible member 370.

The breaking member 320 may further include a securing portion 324 extending from the breaking portion 322 toward the first body portion 312 and spaced apart from the second body portion 314. The frangible member 370 may be disposed between the second body portion 314, the breaking portion 322, and the securing portion 324 prior to the at least one engagement member 360 being received by the socket 140.

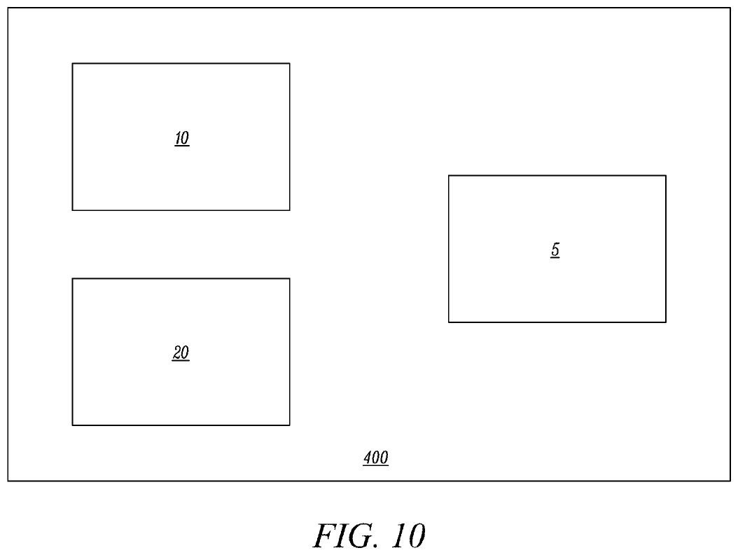

FIG. 10 illustrates a schematic block diagram of a kit 400 according to an embodiment of the present disclosure.

The kit 400 includes the protection device 10. Alternatively, the kit 400 may include the protection device 20 (shown in FIGS. 6A-7). In other words, the kit 400 may include the protection device 10 or the protection device 20. The kit 400 further includes the medical article 5. The protection device 10 may at least partially receive and secure the medical article 5 therein. In some embodiments, the protection device 20 may at least partially receive and secure the medical article 5 therein. Further, the tamper indicating assembly 300 (shown in FIGS. 9A-9C) may indicate tampering with at least one of the protection device 10 and the medical article 5. In some embodiments, the tamper indicating assembly 300 (shown in FIGS. 9A-9C) may indicate tampering with at least one of the protection device 20 and the medical article 5.

The kit 400 may further include gloves, a sterilizing material, a cloth or other absorbent material, and an antiseptic agent. The kit 400 may further include cleaning articles, such as cleaning cloth, cotton balls, cotton swabs, and the like. The kit 400 may be available to a clinician for use in a sterilized package.

The kit 400 may be used with a catheter device (not shown) by the clinician. In some embodiments, the kit 400 may further include the catheter device. The clinician may prevent accidental access or intentional unauthorized access to the catheter device via the medical article 5 by securing the medical article 5 with the protection device 10 or the protection device 20.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A protection device for a medical article, the protection device comprising:
an inner housing comprising:
a first inner end;
a second inner end opposite the first inner end;
a first inner surface defining a first compartment configured to at least partially receive the medical article therein;
a first outer surface opposite the first inner surface; and
at least one inner locking feature disposed on the first outer surface; and
an outer housing comprising:
a first outer end that is open;
a second outer end opposite the first outer end;
a second inner surface defining a second compartment configured to at least partially receive the inner housing therein;
a second outer surface opposite the second inner surface; and
at least one outer locking feature disposed on the second inner surface and complementary to the at least one inner locking feature, wherein the at least one outer locking feature is configured to lockingly engage with the at least one inner locking feature;
wherein the outer housing is configured to at least partially and slidably receive the inner housing therein through the first outer end along a first direction from the first outer end to the second outer end,
wherein the inner housing is insertable within the outer housing from the second inner end,
wherein, upon insertion of the inner housing within the outer housing along the first direction, the at least one inner locking feature lockingly engages with the at least one outer locking feature, and
wherein, upon locking engagement of the at least one inner locking feature with the at least one outer locking feature, the inner housing is secured to the outer housing, such that the inner housing is prevented from moving relative to the outer housing along a second direction opposite to the first direction.

2. The protection device of claim 1, wherein the at least one inner locking feature comprises a protrusion extending from the first outer surface opposite to the first inner surface, and wherein the at least one outer locking feature comprises a recess extending partially from the second inner surface toward the second outer surface and configured to at least partially receive the protrusion therein.

3. The protection device of claim 2, wherein the protrusion comprises a triangular cross-sectional shape, and wherein the recess comprises a complementary triangular cross-sectional shape.

4. The protection device of claim 3, wherein the first outer surface corresponds to a base of the triangular cross-sectional shape, and wherein the other two sides of the triangular cross-sectional shape define different respective angles with respect to the base, such that upon locking engagement of the at least one inner locking feature with the at least one outer locking feature, the inner housing is slidable relative to the outer housing along the first direction and the inner housing is prevented from moving relative to the outer housing along the second direction.

5. The protection device of claim 4, wherein the angle defined by the side of the triangular cross-sectional shape proximal to the second inner end of the inner housing is between 30% to 80% of the angle defined by the side of the triangular cross-sectional shape distal to the second inner end of the inner housing.

6. The protection device of claim 2, wherein the protrusion circumferentially extends around the first outer surface of the inner housing, and wherein the recess circumferentially extends around the second inner surface of the outer housing.

7. The protection device of claim 1, wherein the protection device produces a locking feedback upon locking engagement of the at least one inner locking feature with the at least one outer locking feature.

8. The protection device of claim 7, wherein the locking feedback comprises at least one of an audible feedback and a tactile feedback.

9. The protection device of claim 1, wherein the inner housing further comprises:
a primary portion comprising a first lid locking feature; and
a lid portion movably coupled to the primary portion and comprising a second lid locking feature complementary to the first lid locking feature, wherein the lid portion is movable relative to the primary portion between an open position and a closed position;
wherein the inner housing is configured to at least partially receive the medical article therein in the open position of the lid portion, wherein the first lid locking feature lockingly engages with the second lid locking feature in the closed position of the lid portion, such that the primary portion and the lid portion are secured to each other, wherein the medical article is secured within the inner housing in the closed position of the lid portion, and wherein, upon locking engagement of the at least one inner locking feature with the at least one outer locking feature, the outer housing at least partially encloses the lid portion and retains the lid portion in the closed position.

10. The protection device of claim 9, wherein the inner housing further comprises a hinge pivotally coupling the lid portion to the primary portion.

11. The protection device of claim 10, wherein the hinge is a living hinge.

12. The protection device of claim 9, wherein the first lid locking feature and the second lid locking feature together form a snap-fit connection.

13. The protection device of claim 9, wherein the inner housing comprises an inner housing length, wherein the lid portion comprises a lid length, and wherein the lid length is greater than 50% of the inner housing length.

14. The protection device of claim 1, wherein the second inner end of the inner housing is closed.

15. The protection device of claim 1, wherein the second outer end of the outer housing is closed.

16. The protection device of claim 1, wherein the inner housing comprises an opening disposed at the first inner end, and wherein the opening is configured to at least partially receive the medical article therethrough.

17. The protection device of claim 1, wherein the inner housing further comprises a flange disposed proximal to the first inner end, the flange extending from the first outer surface opposite to the first inner surface, and wherein, upon locking engagement of the at least one inner locking feature with the at least one outer locking feature, the outer housing engages with the flange to prevent further movement of the outer housing relative to the inner housing along the first direction.

18. The protection device of claim 17, wherein the flange extends circumferentially around the first outer surface of the inner housing.

19. The protection device of claim 1, wherein the outer housing further comprises:
a pair of lines of weakness extending along a length of the outer housing and defining a tear region therebetween; and
a tab coupled to the tear region and extending outwardly relative to the second outer surface;
wherein at least the tear region is configured to separate from the rest of the outer housing upon application of force on the tab at least in the first direction.

20. The protection device of claim 1, wherein the inner housing further comprises a first cutout, wherein the outer housing further comprises a second cutout, wherein, upon insertion of the inner housing within the outer housing, the first cutout is at least partially aligned with the second cutout, and wherein the first cutout and the second cutout together at least partially receive the medical article therethrough.

21. The protection device of claim 1, wherein the at least one inner locking feature comprises a first inner locking feature proximal to the first inner end and a second inner locking feature distal to the first inner end, wherein the at least one outer locking feature comprises a first outer locking feature proximal to the first outer end and a second outer locking feature distal to the first outer end, wherein, during insertion of the inner housing within the outer housing along the first direction, the second inner locking feature slides over the first outer locking feature, wherein, upon locking engagement of the first inner locking feature with the first outer locking feature, the second inner locking feature lockingly engages with the second outer locking feature, and wherein, upon locking engagement of the first inner locking feature with the first outer locking feature and the second inner locking feature with the second outer locking feature, the inner housing is prevented from moving relative to the outer housing along the second direction.

22. A kit comprising:
a medical article; and
the protection device of claim 1;
wherein the protection device at least partially receives and secures the medical article therein.

23. A protection device for a medical article, the protection device comprising:
an inner housing comprising:
a first inner end;
a second inner end opposite the first inner end;
a first inner surface defining a first compartment configured to at least partially receive the medical article therein;
a first outer surface opposite the first inner surface;
a first cutout extending from the first inner surface to the first outer surface; and
at least one inner locking feature disposed on the first outer surface; and
an outer housing comprising:
a first outer end and a second outer end opposite the first outer end, wherein the first outer end is open;
a second inner surface defining a second compartment configured to at least partially receive the inner housing therein;
a second outer surface opposite the second inner surface;
a second cutout extending from the second inner surface to the second outer surface, wherein the second cutout partially extends from the first outer end toward the second outer end; and
at least one outer locking feature disposed on the second inner surface and complementary to the at least one inner locking feature, wherein the at least one outer locking feature is configured to lockingly engage with the at least one inner locking feature;
wherein the outer housing is configured to at least partially and slidably receive the inner housing therein through the first outer end along a first direction from the first outer end to the second outer end,
wherein the inner housing is insertable within the outer housing from the second inner end,
wherein, upon insertion of the inner housing within the outer housing, the first cutout is at least partially aligned with the second cutout,
wherein the first cutout and the second cutout together at least partially receive the medical article therethrough,
wherein, upon insertion of the inner housing within the outer housing along the first direction, the at least one inner locking feature lockingly engages with the at least one outer locking feature, and
wherein upon locking engagement of the at least one inner locking feature with the at least one outer locking feature, the inner housing is secured to the outer housing, such that the inner housing is prevented from moving relative to the outer housing along a second direction opposite to the first direction.
